(19) United States
(12) Patent Application Publication
(10) Pub. No.: US 2010/0083960 A1
(43) Pub. Date: Apr. 8, 2010

(54) Activating Mechanism

(75) Inventor: Carlos Guillermo, Los Osos, CA (US)

Correspondence Address: POTOMAC PATENT GROUP PLLC, P. O. BOX 270, FREDERICKSBURG, VA, 22404, US

(73) Assignee: SHL Group AB, Nacka Strand (SE)

(21) Appl. No.: 12/443,847

(22) PCT Filed: Oct. 4, 2007
(86) PCT No.: PCT/EP07/60526

§ 371 Date: Dec. 11, 2009

Related U.S. Application Data
(63) Continuation of application No. 60/828,318, filed on Oct. 5, 2006.

(30) Foreign Application Priority Data
Oct. 5, 2006 (SE) 0602117-4

(51) Int. Cl. A61M 15/00

(52) U.S. Cl. 128/203.12

(57) ABSTRACT

The present invention relates to a Device for a medical delivery device, which medical delivery device comprises a housing (10); a medicament container (22); a canister housing comprising an inhalation opening and means for enabling communication between the inhalation opening and the medicament container; actuating means (26, 27, 30, 32, 34, 72, 74) capable of delivering a dose of medicament, when activated; and activating means capable, upon start of inhalation, to activate said actuating means, said activating means comprising an activating mechanism having at least one enclosure (57), said enclosure having at least one movable wall part (42) and at least one fixed wall part (44), said enclosure being in air flow communication with said inhalation opening, whereby, during inhalation, a pressure drop is created in said enclosure affecting said movable wall to move a certain distance, which in turn activates said actuating means. The invention is characterised in that said movable wall (42) is attached to a movable piston rod (34), which when moved, is capable of activating said actuating means.

### TECHNICAL FIELD

The present invention relates to an activating mechanism and in particular a breath activating mechanism to be used with medicament delivery devices such as powder and aerosol inhalers, nebulisers and the like.

### BACKGROUND ART

For a number of years inhalers have been used to deliver a metered dose of medicament to the respiratory tract of a patient. Basically there are three types of inhalers, adapted for powder medicament, aerosol driven fluid medicament and nebulisers.

The primary design of most of the inhalers are basically the sane for the different forms of medicament; a housing containing a supply of the medicament, a mouthpiece, air flow conduits in connection with the supply of medicament and activating means for generating delivery of a metered dose of medicament. The activating means have a wide variety of constructions and functions. These include activation by the patient's hand, such as squeezing the inhaler or manoeuvring a button, during inhalation, electrically activated dose delivery, or inhalation activated dose delivery, for example.

The trend regarding inhalers, and for that matter, most medicament delivery devices that are intended for self-administration, is to have more and more automatic features, i.e. the patient or user should preferably perform only a few actions in order to have the medicament delivered. One example is disclosed in WO-0078378 where an inhaler is arranged with means for activating a canister, containing medicament, to open and dispense the medicament in response to a user inhaling, which causes an airflow in the inhaler, return means for deactivating said canister to close it, where the return means deactivates said canister when the airflow drops below a certain threshold value.

The primary advantage of the invention according to WO'378 is that the beginning and termination, i.e. activation and deactivation, is controlled by the patient's inhalation and not the device, since the start of the inhalation activates the inhaler to deliver its dose and the end of the inhalation deactivates the inhaler, i.e. closes and refills/recharges it. This in fact increases the inhalation quality by the fact that the canister returns to its decompressed position after completing the inhalation meanwhile the metered dose chamber is refilled. This ensures refilling/recharging of the chamber when the canister is held in a vertical position with the metered dose chamber facing downwards. It's virtually impossible to have an improper refilling/recharging of the chamber when the canister has a low level of medicament, thus ensuring that a correct fill and not propellant gas enters the chamber. The inhaler according to WO'378 could be regarded as breath operated rather than breath activated, as with known inhalers, because both start and end of inhalation activates and deactivates, respectively, the inhaler.

For many inhalers, such as with the inhaler according to WO'378, the activating means is a flap or a vane that is arranged adjacent an air intake on the inhaler and substantially blocking the air intake when not activated. When a patient inhales through an inhalation opening, a pressure difference occurs over the vane or flap. This pressure difference causes the flap or vane to move and thereby open the air intake so that an inhalation air flow is created. This movement of the flap or vane releases the locking means so that the actuating means is activated and a dose is delivered.

For some applications and devices the use of a flap or vane has a few drawbacks. The spring means of the actuating means are often rather powerful. On the other hand the force that the flap or vane can produce in order to activate the actuating means is rather low, which means that there has to be some sort of transmission between the vane and the actuating means in order for the device to function. This in turn means an increased number of components that have to interact with each other in a reliable way in order to ensure delivery of medicament when a user inhales.

GB 1 269 811 discloses an inhaler comprising drive means activated by an activation means for delivering a dose of medicament during inhalation.

The activation mechanism comprises a compartment limited by a movable plate and a fixed wall, which compartment is in flow communication with a mouthpiece of the inhaler. When a user inhales, an under-pressure is created in the compartment, causing the movable plate to be displaced a distance, whereby a linkage arranged to the movable plate affects activation means, which in turn release the drive means so that an inhalation dose is delivered.

The design of GB 1 269 811 requires a rather large movable plate in order for the under pressure to be so large that the plate, and also the linkage, are moved in order to deliver a dose of medicament. Because of the size of the plate, the device becomes large by necessity.

Another drawback with the above described devices is that the breath-activated devices may unintentionally be triggered when the inhaler is ready for inhalation if the inhaler is dropped or otherwise exposed to sudden impact forces. Since the plates, vanes or flaps should be able to move by rather small forces exerted by the pressure drop/air flow during inhalation, they might also rather easily be trigged by a sudden movement or sudden change of movement of the inhaler, such as if the inhaler is shaken or hits an object when it is ready for inhalation.

### DISCLOSURE OF INVENTION

The aim of the present invention is to provide a breath activated mechanism that remedies the above mentioned drawback, in order to ensure a reliable activation of the inhalation device upon inhalation, and that does not lead to a more complicated device and/or provides a more robust mechanism that is generally not affected by external forces.

This aim is achieved according to the features of independent claim 1. Preferable embodiments of the invention are subject of the dependent patent claims.

According to a main aspect of the invention, it is characterised by a device for delivery of medicament, which medical delivery device comprises a housing, an inhalation opening in said housing, a medicament container, means for enabling communication between the inhalation opening and the medicament container, actuating means capable of delivering a dose of medicament, when activated, and activating means capable, upon start of inhalation, to activate said actuating means, characterised in that said activating means comprises an activating mechanism having at least one enclosure, said enclosure having a movable wall part and a fixed wall part, said enclosure being in air flow communication with said inhalation opening, whereby, during inhalation, a pressure drop is created in said enclosure affecting said movable wall to move a certain distance, which in turn activates said actuating means.

According to another aspect of the invention, it is characterised in that said enclosure comprises one movable disk and a fixed disk interconnected by a flexible gas-tight membrane. Preferably the movable disk is attached to a movable piston rod, which when moved, is capable of activating said actuating means.

According to yet another aspect of the invention, it is characterised in that a number of enclosures are interconnected to each other in a manner to increase the moving force due to the pressure drop.

According to a further aspect of the invention, it is characterised in that said set of disks are arranged in an trigger housing disk chamber, said trigger housing disk chamber being in air flow communication with said inhalation opening. Further said fixed disk is preferably arranged with a flange having a number of openings, thus providing air flow communication between the trigger housing disk chamber and said enclosure. Also an air flow communication passage is arranged between each set of disks and the surrounding air, for preventing pressure drop between the sets during movement of the movable disks.

According to a further aspect of the invention, it is characterised in that it further comprises manual activation means, arranged and designed, upon manual operation, to move said movable wall for activating said actuating means.

There are a number of advantages with the device according to the present invention. By the design of having an enclosure with one movable and one fixed wall, in which enclosure pressure drop is created during inhalation, a very compact, reliable and safe activation mechanism for a medical delivery device is obtained. The compact aspect is important for many delivery devices in order to keep the size of the delivery device within reasonable and acceptable dimensions. Often the users do not want to show too much that they are using this kind of device so the size aspect can be important. Further, the risk of unintentional activation of the delivery device caused by external impact forces is reduced in comparison to the vane or flap type activating mechanisms used in many inhalation delivery devices.

According to a preferred embodiment of the invention, the enclosures are formed by a set of disks where one is fixed and the other is movable, which is a space-saving design. The fact that it is possible to arrange a number of sets of disks interacting with each other provides the possibility of increasing the activation force from the inhalation, i.e. increasing the active surface of the activating mechanism and at the same time keeping it compact.

Regarding many inhalers that require priming before the first inhalation of a user, the activating mechanism according to the present invention, is arranged with means for manually activating the mechanism and thereby releasing a dose of medicament.

The device according to the present invention is suitable in a wide range of medicament delivery devices that use the inhalation to activate a function, e.g. inhalation activation and inhalation operation. In this respect the medicament could be in powder form, in an aerosolizing suspension, in a nebuliser unit. It is also to be understood that the inhalation could be performed by the mouth or by the nose of a user.

These and other aspects of and advantages with the present invention will become apparent from the following detailed description and from the accompanying drawings.

### BRIEF DESCRIPTION OF DRAWINGS

In the following detailed description of the invention, reference will be made to the accompanying drawings, of which

FIG. 1 shows an example of an inhaler in cross-section comprising the device according to the invention,

FIG. 2 shows a perspective view of the inhaler according to FIG. 1 without an inhaler housing,

FIG. 3 shows a detailed view of a part of the device according to FIG. 1 in perspective cross-section,

FIG. 4 shows a side view of the activating mechanism according to the present invention,

FIG. 5 shows the activating mechanism according to FIG. 4 in a top view,

FIG. 6 shows the activating mechanism according to FIGS. 4 and 5 in a cross-sectional side view,

FIG. 7 shows a detailed view of a part of the activating mechanism of FIGS. 4-6, and

FIGS. 8a-b show an alternative medicament delivery device comprising the present invention.

### DETAILED DESCRIPTION OF THE INVENTION

The activating mechanism according to the present invention is intended to be used with breath-activated or -operated medicament delivery devices such as inhalers, nebulisers and the like.

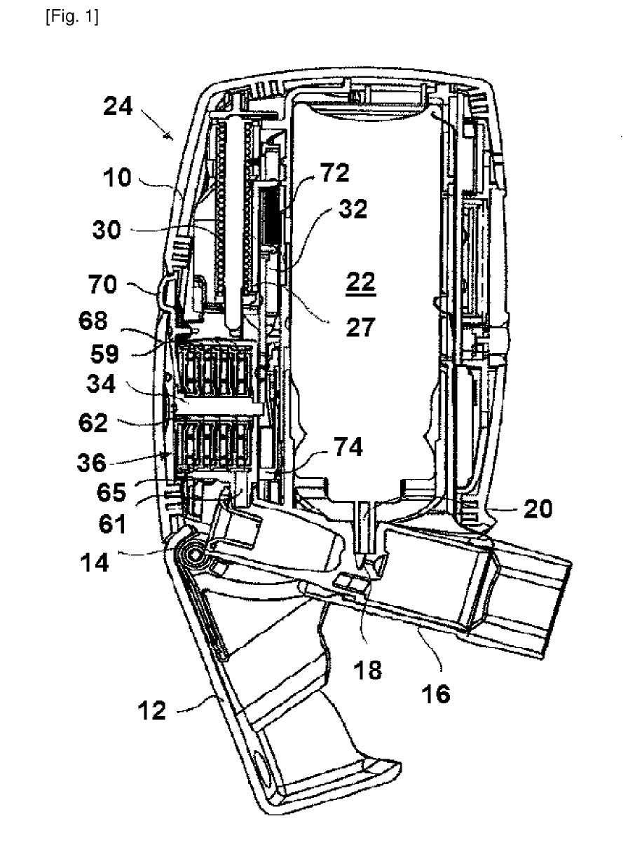

FIG. 1 shows an example of an inhaler comprising the present invention. The inhaler according to FIG. 1 comprises a housing 10. The lower part of the housing is arranged with a lid 12 which is pivotable around a pivot point 14 between a closed position and an open position, as shown in FIG. 1. Further, inside the lower part of the housing is arranged a canister housing which comprises a slidable mouth piece 16, an aerosolizing chamber 18, a channel and a nozzle, wherein said mouth piece extends and protrudes outside the housing when the lid is opened, due to a telescopic spring loaded design.

In the mouth-piece channel the aerosolizing chamber 18 is arranged with an outlet facing the mouth-piece opening. The aerosolizing chamber communicates with the stem 20 of an aerosol canister 22 containing medicament. The canister is arranged slidable in the housing to be able to depress the canister stem into the canister in order to deliver a dose of medicament, which is aerosolized in the aerosolizing chamber before exiting through the outlet.

A trigger housing 28 is also arranged inside the housing. Said trigger housing comprises a trigger housing disk chamber 38 and a channel 61 which is in flow communication with the interior of the mouth piece.

An actuating means 24 are also arranged in the housing, capable of depressing the canister when activated. It comprises a canister bracket 26, a plunger 27, a plunger spring 30 a release shuttle 32, a piston rod 34, a reset link 74 and a reset spring 72. Said canister bracket 26, in which the top of the canister is situated, extends with a rear part into the trigger housing in such a way that the canister bracket is shade in the trigger housing 28 in a vertical direction. The plunger 27 comprises upper arms which interact with the canister bracket, lower arms which interact with the trigger housing and a flexible tab which interacts with the release shuttle 32. A plunger spring 30 is arranged between an upper part of the trigger housing and a lower end of the plunger 27. Further, when the inhaler is a non activated state or when the lid 12 is closed, cams on the lid 12 which interacts with the reset link 74, holds the reset link upwards. Meanwhile, said reset link 74 supports the plunger 27 against the plunger spring force. While the plunger 27 is suspended, the release shuttle is pulled upwards via the reset spring 72, because the plunger arms are relieved from contact against the canister bracket and trigger housing. All contact and loads in the sensitive trigger chain are relieved while the lid 12 is closed. Any motion of the piston rod will not trigger activation because the release shuttle is suspended off of the piston rod. The last several degrees of closing the lid will therefore always be supporting the plunger spring force, and this energy is released when the lid is opened.

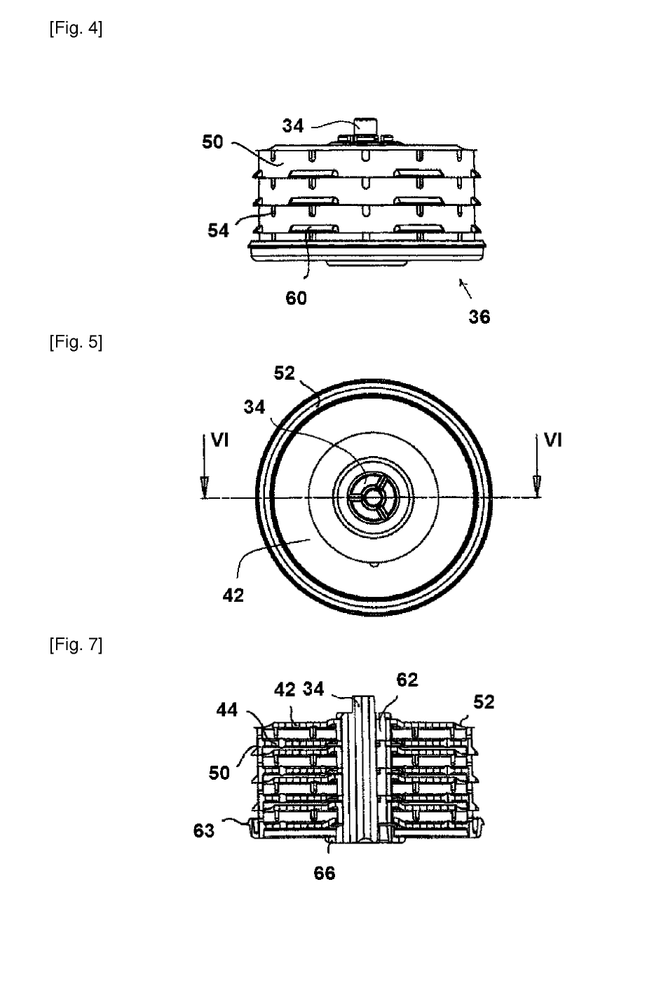

The piston rod 34 is also a component in the activating mechanism 36 of the present invention. It extends through an opening in an end wall of the trigger housing disk chamber 38. The opposite end of the trigger housing disk chamber is not provided with an end wall but is open to the rear inner wall of the housing. A breath activating means is arranged in said trigger housing disk chamber, where components of said activating means are attached to said piston rod 34. The activating means is arranged as a number of disks stacked after each other in the axial direction of the piston rod, FIGS. 4-6, where each second disk 42, hereafter named piston disk, is attached to the piston rod 34 and each second disk 44, hereafter named cylinder disk, is held in a fixed position in relation to the trigger housing disk chamber, as will be described below. Each set of one disk attached to the plunger rod and one fixed disk forms a part of the breath activating mechanism.

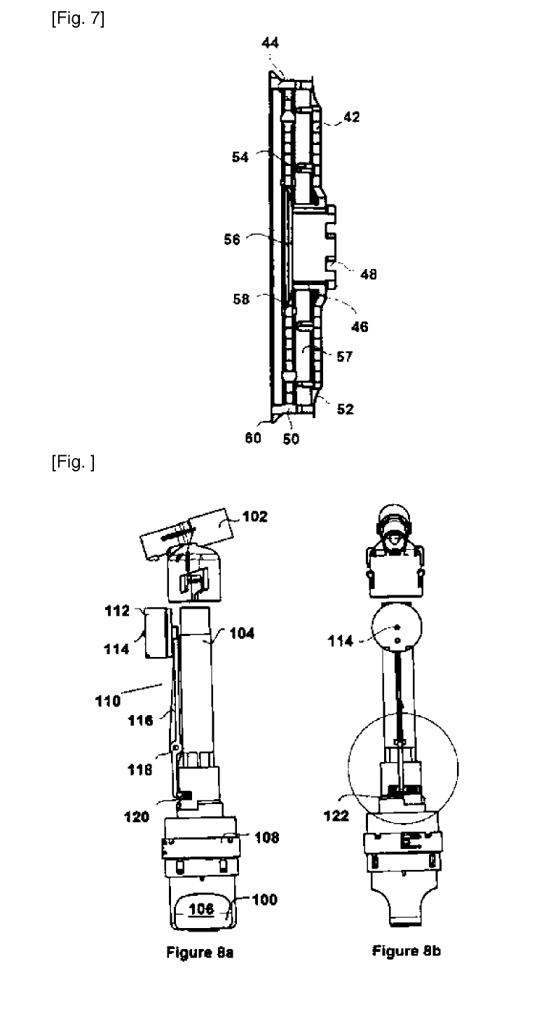

FIG. 7 shows such a set of disks. The piston disk 42 to the right in the figure is attached to the piston rod 34 via a cylindrical hub 46. The right end of the hub is arranged with cut-outs 48, the function of which will be described below. The cylinder disk 44 to the left is arranged with a flange 50 around its circumference extending on both sides of the disk. A gas-tight flexible membrane 52 is attached to the outer circumference of the right disk 42 and to the right edge of the flange 50. Further, the right part of the flange is arranged with a number of openings 54 around the circumference. The centre of the left cylinder disk 44 is arranged with a central circular opening 56. Between the edge of the circular opening 56 and the edge of the hub 46 facing to the left in the figure, a flexible membrane 58 is arranged.

Thus an annular enclosure 57 is arranged between the disks 42, 44 and delimited by the flexible membranes 52, 58 and the flange 50 and with air communication passages through the openings 54 in the flange in a manner that will be described below. The outer surface of the flange is further arranged with distance pieces 60 that are in contact with the inner wall of the trigger housing disk chamber. There is thus a space 59 formed between the trigger housing disk chamber and the disks. This space is in flow communication with the interior of the mouth-piece via channel 61. Further a longitudinal groove 62 is arranged in the piston rod 34 which is in air flow communication with a space formed between two sets of disks via the cut-outs 48 in the hub. The sets of disks are “stacked” one set after the other where the flanges are abutting each other. The last fixed disk, the leftmost in the figures, is arranged with an annular protrusion 63, FIG. 6, which, when the mechanism is inserted into the trigger housing disk chamber, snap-fits into an annular groove 65, FIG. 1, in the inner wall of the chamber in an air-tight manner, holding all the fixed disks in place inside the chamber.

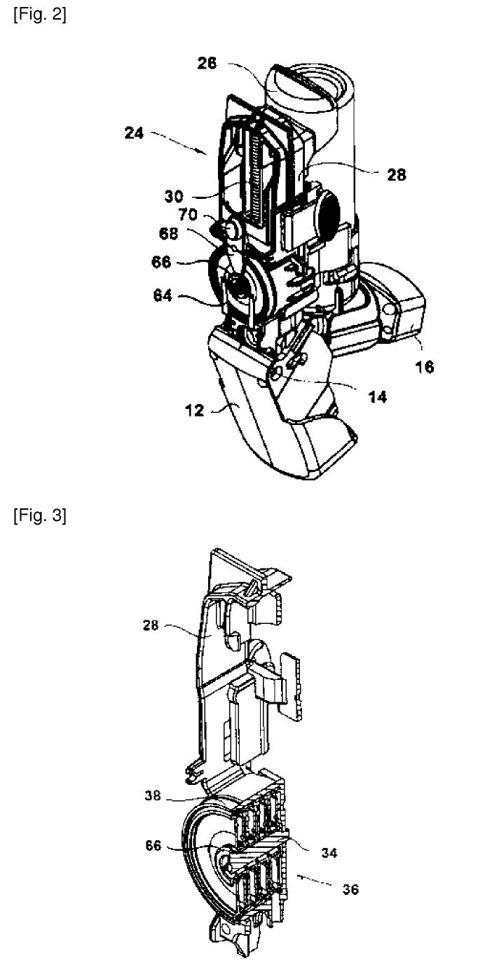

Return means 64, FIG. 2, attached to the outer surface of the trigger housing disk chamber, in the form of a piston return spring is acting on the left end surface of the piston rod, urging it to the right in the figures. The left end of the piston rod is arranged with an annular ledge 66. A manual activating mechanism is arranged adjacent the piston rod. It comprises an arm 68 pivotally arranged about its mid-point. One end of the arm is in contact with the ledge of the piston rod, and the other end of the arm is arranged with a protrusion 70 extending through the housing of the device, thereby acting as an activation button. This activation button can be used as a priming button but also as an emergency button in cases when the patient has not the pulmonary capacity to activate said inhaler.

The device is arranged to function as follows. When the lid 12 is opened the mouth-piece 16 extends and the device is ready for medicament delivery. The opening of the lid lowers the reset link 74, which allows the plunger arms to be loaded onto the trigger housing ledges, supporting the force of the plunger spring. The plunger arms are biased inward due to a ramp angle on the trigger housing, and rest against the ramps of the release shuttle. This causes the release shuttle 32 to move downward with a light force, acting against the force of the reset spring 72, and then resting onto the piston rod 34. If a new aerosol canister has been installed in the device, it must be primed at least one time before use. The manual activation button 70 extending through the housing is then depressed. This causes the arm 68 of the piston return spring to pivot and because the end of the arm is in contact with the ledge 66 of the piston rod 34, it urges the piston rod in the longitudinal direction to the left in the figures. Displacement of piston rod releases the release shuttle which slides down to a position until engaging the flexible tab on the plunger. This allows the plunger lower arms to collapse inwards out of engagement with the trigger housing, and the plunger is then pushed downward by the plunger spring. Meanwhile, the release shuttle has moved into a position to block the upper arms of the plunger, which engage the canister bracket, causing it to move with the plunger, thus compressing the canister, whereby the canister stem is depressed. This in turn causes a metered dose to be released into the aerosolizing chamber 18 and then into the mouth-piece 16 and into the air. When the activation button is released the piston return spring 64 urges the piston rod 34 back to its original position, to the right in the figures, whereby it pushes the plunger flexible tip out of the way of the release shuttle. This allows the release shuttle to continue downward. Thereafter the reset spring pushes upwards the canister bracket, whose ramped ledges cause the upper plunger arms to bend inward, allowing the canister bracket to be disengaged from the upper plunger arms. This relieves all loads from the canister, which in turn is urging the canister bracket and the canister upwards. When the lid is closed it acts on the reset link 74 to put the system in order for a subsequent dose. During closing of the lid, the reset link urges the plunger back to its initial position against the force of the plunger spring, at which position the release shuttle is freed to move to its first position by the force of the reset spring 72.

When a patient or user is to take a dose the lid is again opened and the mouth-piece extends. The user then inhales in the mouth-piece, which creates an air flow in the mouthpiece. The suction action from the inhalation creates an inhalation suction pressure. Said pressure is transferred through the canister housing channel, which is in a sealed connection with channel 61, into the trigger housing disk chamber and further into the space 59 surrounding the disks and in the annular enclosure 57 between the disks due to the flow communication via the openings 54. Said suction pressure is amplified ˜40% by venturi effect through the nozzle of the spray head. The nozzle diameter is sized to obtain desired flow vs. pressure drop. Suction pressure evacuates the disk subassembly, applying a pressure drop to at least a set of piston disks. The piston rod is pulled back with piston disks once sufficient pressure develops to overcome the piston return spring. Due to the membranes (52, 58), none leakage appears and then the pressure drop is used in its most effective way. In order to ensure that there is no pressure drop in the enclosure between the adjacent set of disks due to the movement, this space is in air flow communication with interior of the device via the groove 62 in the piston rod and the cut-outs 48 in the hubs 46. The moving force depends on the total disk area and is thereby amplified proportionally to the number of sets of disks which are arranged after each other, acting in the same manner and simultaneously. The movement of the piston rod causes it to move out of contact with the release shuttle, whereby the same procedure is performed as described above in connection with the priming.

FIGS. 8a-b show an alternative medicament delivery device, in this case a nebuliser, where its actuation means generally corresponds to the device described in patent application PCT/SE2006/050150. The device shown has an outer housing removed for clarity. The device comprises in its distal end a dose setting member in the form of a dose wheel 100 and in its proximal end a mouth piece 102, connected to a cartridge 104. The cartridge 104 is intended to be filled with the liquid medicament to be administered to the patient. The mouth piece is further arranged with a porous membrane, which is arranged in a channel in communication with the interior of the cartridge. The membrane is preferably a Rayleigh type membrane having pores with a diameter in the range of about 0.25 to 6 pm. The specific dimension and shapes of the pores should be selected to suit the specific pharmaceutical drug.

The dose wheel 100 comprises a dose wheel turning member 106 and a member 108 housing the energy accumulating member (see below), which members 106, 108 are adapted in order to be firmly, and removably, connected with each other. An elongated screw threaded plunger rod (not shown) is running along the longitudinal axis of said device, which device thus is provided with means in order to house such a screw threaded member. The plunger rod is in its proximal end adapted to be in contact with a piston, which piston is sealingly and slidably provided inside the cartridge 104.

The housing member 108 is adapted to house an energy accumulating member in the form of a flat spiral spring, which spring is provided winded in layers around the exterior of the proximal part of the housing member. The flat spiral spring is in its inner end provided with inner holding means in order to be attached to the housing member 108, such as for instance a protruding part adapted to be fitted with a corresponding slit in the housing member 108, or alternatively a hole of a suitable size in the flat spiral spring, and a smaller screw or other similar means for the anchoring of the flat spiral spring in the housing member.

The dose wheel 100 is further in its proximal end adapted to house a coupling member. Said coupling member is in its proximal end provided with a crown, which proximal end in turn is provided with at least one, preferably two equally distributed, bevelled protrusions. The crown of the coupling member is adapted to engage a plunger rod driving member, in the form of a nut. Herein the term nut is defined as a member provided with a through going hole, such that the interior of the member is provided with a thread of predetermined pitch of grooving, i.e. a predetermined screw pitch, said member is thus adapted to be screw threaded on a second member provided with a corresponding thread. The nut is in this case adapted to engage with the plunger rod, i.e. the interior of the nut is provided with grooves of a predetermined pitch in order to be screw threaded on the plunger rod. The nut is designed as to in its proximal end be provided with outwardly protruding flanges and in its distal end be provided with a skirt, the distal end of which is provided with a number of equally distributed bevelled recesses, which correspond to the protrusions of the crown. The coupling member is further provided with a helical coupling spring, which proximal end is in contact with the distal end of the crown. The distal end of the coupling spring is firmly fixed to the coupling member.

Further, the device is arranged with activating means 110. It comprises a trigger housing disk chamber 112 with disk packs and a piston rod 114 as described above. The piston rod is in one end in contact with one arm of a lever 116, which lever is pivotally arranged around a pivot point 118. The opposite arm of the lever is arranged with a number of inwardly directed protrusions or teeth 120, which teeth are in engagement with a toothed outer surface of an actuation member 122.

The device is intended to function as follows. The predetermined dose set by the use of the dose wheel 100, with the use of which the dose is increased by predetermined equally large dose increment steps. One predetermined dose increment step, corresponds to a clock-wise rotation of the dose wheel 100 with one step, which step corresponds to a predetermined number of degrees. Thus, with each dose increment step, the dose wheel turning member is turned clock-wise an additional step corresponding to said predetermined number of degrees.

So, in order to set a predetermined dose that corresponds to for instance two dose increment steps, the dose wheel turning member is turned clock-wise two steps.

When the dose wheel turning member is rotated, the housing member and the coupling member will rotate correspondingly and hence also the inner holding means of the flat spiral spring. Also the shoulder of the housing member will be brought out of engagement with the corresponding means of the outer cover when the housing member is rotated clock-wise.

When the coupling member rotates clock-wise, the protrusions will move along the bevelled edge of the recesses of the plunger rod driving member, with which recesses the protrusions are initially in engage with, and the coupling member will thus move towards the distal end of the delivery device, compress the coupling spring, and unlock the dose wheel from the plunger rod driving member. The flat spiral spring is hereby free to wind up and accumulate energy corresponding to the rotation of the dose wheel turning member the number of degrees corresponding to one clock-wise step turn. Due to the power accumulated in the compressed coupling spring, the coupling member will now move back towards the proximal end of the delivery device when the protrusions climb over the edge of the bevelled recesses and lock the coupling member as well as the dose wheel to the plunger rod driving member, when the protrusions engage the recesses following the recesses it was previously in engagement with. The dose wheel turning member is turned the additional and final step, whereby the above described procedure is repeated. The flat spiral spring has thus accumulated the energy that corresponds to the rotation of the dose wheel turning member the number of degrees corresponding to a two step clock-wise turn.

The delivery device is now ready to deliver the predetermined dose corresponding to two dose increment steps. When a patient inhales in the mouth piece 102 a pressure drop is created in the activating means 110 as described above. This causes the piston rod 114 to move in its longitudinal direction, and thereby push on the arm of the lever 116. This in turn causes the lever to pivot around its pivot point 118, whereby the inwardly directed teeth 120 move out of contact with the teeth of the actuation member 122. When the dose actuation member is activated, said member will bring the external means of the plunger rod driving member out of engagement and the plunger rod driving member is released for rotation.

Due to the energy accumulated in the flat spiral spring in the first dose delivery step, the coupling member and the plunger rod driving member will due to the output torque of the spring when said spring now is free to unwind, rotate counter clock-wise the number of degrees corresponding to the two step turn. The rotation causes the plunger rod to move forward and pressurize the liquid in the container, whereby the liquid is pressed through the porous membrane. Outside of the porous membrane Rayleigh type droplets are formed to be emitted into the airflow of the mouth piece.

When the patient stops inhaling the pressure in the activating means becomes normal and the lever is returned to its original position by a return spring (not shown). This causes the teeth of the lever to come into contact with the teeth of the actuation member.

It is to be understood that the embodiments described above and shown in the drawings are to be regarded only as a non-limiting example of the invention and that it may be modified within the scope of protection of the patent claims.

1.-10. (canceled)

11. An activating mechanism for a medical delivery device that includes a housing; a medicament container; a canister housing, including an inhalation opening and means for enabling communication between the inhalation opening and the medicament container; and an actuating device capable of delivering a dose of medicament when activated; wherein the activating mechanism is capable, upon start of inhalation, of activating the actuating means, and comprises:
at least one enclosure that includes at least one movable wall part and at least one fixed wall part interconnected by flexible gas-tight membranes;
wherein the at least one movable wall is attached to a movable piston rod, which when moved, is capable of activating the actuating means; and the at least one enclosure is in air flow communication with the inhalation opening;
whereby during inhalation, a pressure drop is created in the enclosure affecting the movable wall to move a certain distance, which in turn activates the actuating means.

12. The activating mechanism of claim 11, further comprising a return device capable of returning the at least one movable wall to its original position when the pressure drop terminates.

13. The activating mechanism of claim 11, wherein the area of the at least one enclosure is increased in order to increase a moving force due to the pressure drop.

14. The activating mechanism of claim 11, wherein a number of enclosures are interconnected to each other as a set of walls in order to increase a moving force due to the pressure drop.

15. The activating mechanism of claim 14, wherein the set of walls is arranged in a trigger housing disk chamber that is in air flow communication with the inhalation opening by a channel.

16. The activating mechanism of claim 15, wherein the fixed wall part is arranged with a flange having a number of openings around its circumference and providing air flow communication between the trigger housing disk chamber and the at least one enclosure.

17. The activating mechanism of claim 14, further comprising an air flow communication passage between each set of walls and the surrounding air that prevents pressure drop between the sets during movement of the movable walls.

18. The activating mechanism of claim 11, further comprising manual activating means arranged to move, upon manual operation, the piston rod for activating the actuating means.

19. The activating mechanism of claim 12, wherein the area of the at least one enclosure is increased in order to increase a moving force due to the pressure drop.

20. The activating mechanism of claim 12, wherein a number of enclosures are interconnected to each other as a set of walls in order to increase a moving force due to the pressure drop.

21. The activating mechanism of claim 20, wherein the set of walls is arranged in a trigger housing disk chamber that is in air flow communication with the inhalation opening by a channel.

22. The activating mechanism of claim 21, wherein the fixed wall part is arranged with a flange having a number of openings around its circumference and providing air flow communication between the trigger housing disk chamber and the at least one enclosure.

23. The activating mechanism of claim 13, wherein a number of enclosures are interconnected to each other as a set of walls in order to increase a moving force due to the pressure drop.

24. The activating mechanism of claim 23, wherein the set of walls is arranged in a trigger housing disk chamber that is in air flow communication with the inhalation opening by a channel.

25. The activating mechanism of claim 24, wherein the fixed wall part is arranged with a flange having a number of openings around its circumference and providing air flow communication between the trigger housing disk chamber and the at least one enclosure.

26. The activating mechanism of claim 19, wherein a number of enclosures are interconnected to each other as a set of walls in order to increase a moving force due to the pressure drop.

27. The activating mechanism of claim 26, wherein the set of walls is arranged in a trigger housing disk chamber that is in air flow communication with the inhalation opening by a channel.

28. The activating mechanism of claim 27, wherein the fixed wall part is arranged with a flange having a number of openings around its circumference and providing air flow communication between the trigger housing disk chamber and the at least one enclosure.

29. The activating mechanism of claim 15, further comprising an air flow communication passage between each set of walls and the surrounding air that prevents pressure drop between the sets during movement of the movable walls.

30. The activating mechanism of claim 16, further comprising an air flow communication passage between each set of walls and the surrounding air that prevents pressure drop between the sets during movement of the movable walls.
